(19) United States
(12) Patent Application Publication
(10) Pub. No.: US 2025/0138218 A1
(43) Pub. Date: May 1, 2025

(54) CONNECTION DETECTING SYSTEMS FOR A SURGICAL CONSOLE

(71) Applicant: Alcon Inc., Fribourg (CH)

(72) Inventors: Jiansheng ZHOU, Irvine, CA (US); Kyle Randall LEONG, Tustin, CA (US)

(21) Appl. No.: 18/901,601

(22) Filed: Sep. 30, 2024

Related U.S. Application Data
(60) Provisional application No. 63/594,926, filed on Oct. 31, 2023.

(51) Int. Cl. G01V 8/12; A61B 17/00

(52) U.S. Cl. CPC G01V 8/12; A61B 17/00; A61B 2017/00199; A61B 2017/00477

(57) ABSTRACT

Embodiments disclosed herein provide a connection detecting system for a surgical console. The connection detection system comprises a port for receiving a connector of a surgical device, an emitter, a detector, and a controller connected to the emitter and the detector. The port comprises a light transmission system comprising a detection zone through which the connector of the surgical device is configured to be disposed when the surgical device is coupled to the surgical console, and a pair of light pipes disposed at opposite ends of the detection zone. The emitter is configured to generate a light for propagation through the light transmission system, and the detector is configured to detect the light propagated through the light transmission system. The controller is configured to determine whether the surgical device is connected to the port based on whether the light is detected by the detector.

### CROSS-REFERENCE TO RELATED APPLICATIONS

This application claims the benefit of U.S. Provisional Application Ser. No. 63/594,926 (filed on Oct. 31, 2023), the content of which is incorporated herein by reference in its entirety.

### INTRODUCTION

In preparation for various types of ophthalmic procedures, one or more surgical devices may be connected to a surgical console. Surgical consoles used for ophthalmic procedures typically include one or more connection detecting systems that can identify when a surgical device is connected to the surgical console and/or what type of surgical device is connected to the surgical console.

### SUMMARY

The present disclosure relates generally to device connection detecting systems for surgical consoles.

In certain embodiments, a connection detecting system for a surgical console is provided. The connection detecting system includes a port for receiving a connector of a surgical device, the port comprising a light transmission system comprising a detection zone through which the connector of the surgical device is configured to be disposed when the surgical device is coupled to the surgical console, and a pair of light pipes disposed at opposite ends of the detection zone, where a longitudinal axis of each of the light pipes is parallel relative to a longitudinal axis of the port, and an emitter configured to generate a light for propagation through the light transmission system, a detector configured to detect the light propagated through the light transmission system, and a controller, connected to the emitter and the detector, and configured to determine whether the surgical device is connected to the port based on whether the light is detected by the detector.

In certain embodiments, another connection detecting system for a surgical console is provided. The connection detecting system includes a port for receiving a connector of a surgical device, the port comprising a light transmission system comprising a detection zone through which the connector of the surgical device is configured to be disposed when the surgical device is coupled to the surgical console, and a pair of light pipes disposed at opposite ends of the detection zone, where a longitudinal axis of each of the light pipes is orthogonal to a longitudinal axis of the port, and an emitter configured to generate a light for propagation through the light transmission system, a detector configured detect the light propagated through the light transmission system, and a controller, connected to the emitter and the detector, and configured to determine whether the surgical device is connected to the port based on whether the light is detected by the detector.

The following description and the related drawings set forth in detail certain illustrative features of one or more embodiments.

### BRIEF DESCRIPTION OF THE DRAWINGS

The appended figures depict certain aspects of one or more disclosed embodiments and are therefore not to be considered limiting of the scope of this disclosure.

FIG. 1A illustrates an example of an ophthalmic surgical system that may be used to perform ophthalmic procedures on an eye, according to certain embodiments.

FIG. 1B is an enlarged view illustrating a port interface of the ophthalmic surgical system seen in FIG. 1A, according to certain embodiments.

FIG. 1C is an example of subsystems of a console of the ophthalmic surgical system of FIG. 1A, according to certain embodiments.

FIG. 2A is a side isometric view of an example port of the ophthalmic surgical console of FIG. 1A, according to certain embodiments.

FIG. 2B is an exploded side isometric view of the example port seen in FIG. 2A, according to certain embodiments.

FIG. 2C is a side isometric view of a light pipe of the example port seen in FIG. 2B, according to certain embodiments.

FIGS. 2D-2E are opposing side isometric views of an example printed circuit board (PCB), according to certain embodiments.

FIG. 2F is a cross-sectional isometric view of an example connection detecting system configured to connect with a surgical device, according to certain embodiments.

FIG. 2G is a cross-sectional isometric view of the example connection detecting system of FIG. 2F connected with a connector of a surgical device, according to certain embodiments.

FIG. 3A is a side isometric view of another example port of the ophthalmic surgical console of FIG. 1A, according to certain embodiments.

FIG. 3B is an exploded side isometric view of the example port seen in FIG. 3A, according to certain embodiments.

FIG. 3C is a first side isometric view of another example PCB, according to certain embodiments.

FIG. 3D is a cross-sectional isometric view of another example connection detecting system configured to connect with a surgical device, according to certain embodiments.

FIG. 3E is a cross-sectional isometric view of the example connection detecting system of FIG. 3D connected with a connector of a surgical device, according to certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

It will be readily understood that the components of the embodiments as generally described herein and illustrated in the appended Figures can be arranged and designed in a wide variety of different configurations. Thus, the following more detailed description of various embodiments, as represented in the Figures, is not intended to limit the scope of the present disclosure but is merely representative of various embodiments. While the various aspects of the embodiments are presented in the Figures, the Figures are not necessarily drawn to scale unless specifically indicated.

Reference throughout this specification to the term “distal” refers to a system, device, component, end, portion, or segment that is disposed closer to a patient and/or further from a console during an ophthalmic procedure; and the term “proximal” refers to the system, device, component, end, portion, or segment that is disposed further from the patient and/or closer to the console during the ophthalmic procedure.

Current connection detecting systems of surgical consoles often employ Radio-Frequency Identifier (RFID) and/or pressure pulse technologies to determine whether surgical devices are connected to a console. For example, when a connector of a surgical device is connected to a port of a surgical console, an RFID reader within the surgical console may sense that an RFID tag of the connector is nearby, thereby allowing the system to determine that a device has been connected thereto. In some examples, to determine proper connection of a pneumatically-driven surgical device to a console, one or more pressure pulses from a pneumatic source may be sent to the port when initiating operations of the pneumatically-driven surgical device. The pressure pulse(s) may be measured, using a port pressure sensor, to determine if pressure has built up at the port, as caused by a connector being inserted into the port. The absence of pressure buildup signals that no surgical device is currently connected to the port. RFID and pressure pulse mechanisms may be employed independently or combined in a sequential manner. For example, an RFID reader may be utilized to detect the presence of a connector and a device type, and after initiation of device operations, pressure pulses may be utilized to confirm proper connection of the device.

Yet, current RFID connection detecting systems may be inconsistent, since proper engagement with surgical devices is not ensured due to an inability to detect whether a connector is completely engaged with a port, i.e., whether there is a gap between the connector and the port or not. Further, current pressure pulse systems may only allow for one-time detection and may not continuously monitor the connection between the connector of the surgical device and the port. As such, current connection detecting systems present a variety of limitations that increase the risk for faults during surgical procedures when the surgical device is not securely connected and/or becomes disconnected. In such cases, various patient-related complications can occur.

Accordingly, the systems described herein overcome many of the limitations associated with connection detecting systems.

Certain embodiments described herein provide improved connection detecting systems for use during the preparation for, and performance of ophthalmic procedures. More particularly, certain embodiments provide connection detecting systems that continuously and more accurately detect and monitor proper connection of surgical devices to a surgical console, which reduces complications associated with improper connections, surgical device faults, or other similar events affecting the surgical devices and or the surgical console during ophthalmic procedures.

Certain embodiments of the present disclosure are directed to a connection detecting system. In some embodiments, the system includes a port for receiving a connector of a surgical device, an emitter, a detector, and a controller connected to the emitter and the detector. The port includes a light transmission system with a detection zone through which the connector of the surgical device is configured to be disposed when the surgical device is coupled to the surgical console, and a pair of light pipes disposed at opposite ends of the detection zone. The emitter is configured to generate a light for propagation through the light transmission system, and the detector is configured to detect the light propagated through the light transmission system. The controller is configured to determine whether the surgical device is connected to the port based on whether the light is detected by the detector.

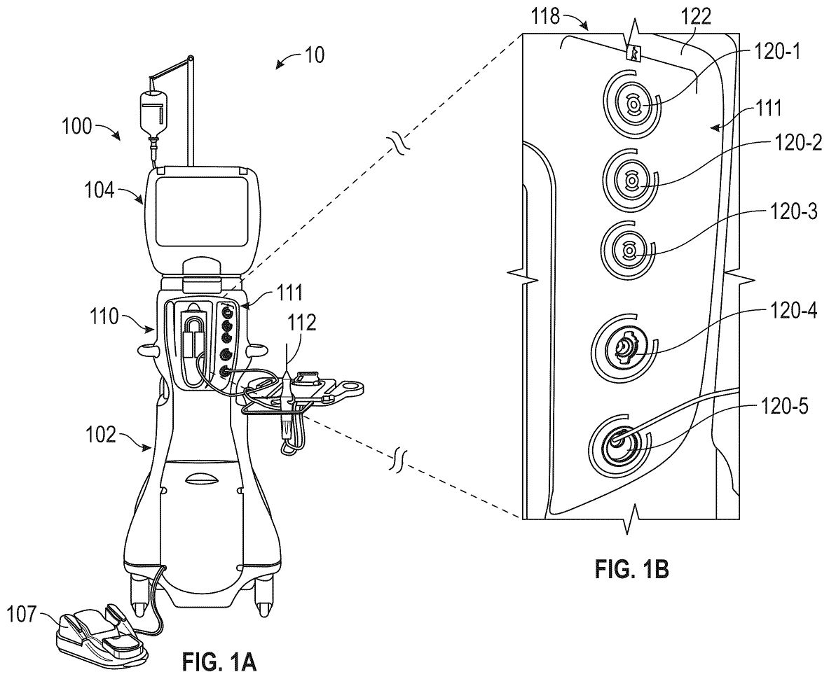

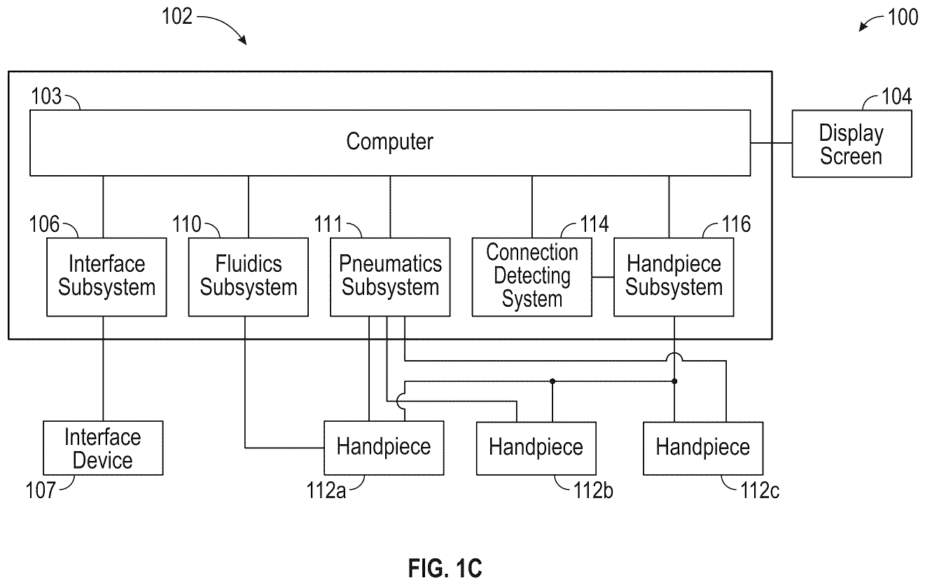

FIG. 1A illustrates an example of an ophthalmic surgical system 10 that may be used to perform ophthalmic procedures on an eye, according to certain embodiments. In the illustrated embodiments, system 10 includes a console 100 (also referred to as a “surgical console”), an interface device 107 (e.g., a foot pedal or footswitch), and a handpiece 112. Console 100 includes a housing 102, a display screen 104, a fluidics subsystem 110, and a pneumatics subsystem 111. The components of system 10 and console 100 may be coupled as shown and are described in more detail with reference to FIG. 1B.

FIG. 1B is an enlarged view illustrating a port interface 118 of the ophthalmic surgical system seen in FIG. 1A, according to certain embodiments. The port interface includes an external face 122 with a plurality of ports: a first port 120-1, a second port 120-2, a third port 120-3, a fourth port 120-4, and a fifth port 120-5 (collectively referred to herein as “plurality of ports 120”).

The plurality of ports 120 may be configured to couple with connectors of a plurality of handpieces and/or other devices (e.g., surgical devices). For example, the first port 120-1 may be configured to connect with a viscous fluid control (VFC) connector, the second port 120-2 may be configured to connect with a forceps connector, the third port 120-3 may be configured to connect with a scissors connector, the fourth port 120-4 may be configured to connect with a low pressure air source (LPAS) connector, and the fifth port 120-5 may be configured to connect with a vitrectomy probe connector.

Although the port interface 118 is shown as including five ports 120, the port interface 118 may include less than five ports or more than five ports. In addition, more than or less than five ports may be configured to connect with the same or different types of connectors and/or devices. Further, although the five ports 120 are shown as part of the pneumatics subsystem 111, which is configured to connect with pneumatic driven handpieces, in the illustrated embodiments, other types of subsystem ports may be configured to connect with other types of handpieces and/or other devices via ports 120 in the port interface 118. For example, an illuminator subsystem port may be configured to connect with an illumination handpiece.

FIG. 1C illustrates example subsystems of console 100 of ophthalmic surgical system 10 of FIG. 1A, according to certain embodiments. Console 100 includes housing 102, which accommodates a computer 103 (with an associated display screen 104) and subsystems 106, 110, 111, and 116, which support interface device 107 and handpieces 112 (112a-c). An interface device 107 receives input to console 100, sends output from console 100, and/or processes the input and/or output. Examples of an interface device 107 may include a foot pedal, another type of manual user input device (e.g., a keyboard), and/or a display. Interface subsystem 106 receives input from and/or sends output to interface device 107.

Computer 103 controls operation of ophthalmic surgical system 10. Generally, computer 103 includes a processor and a memory. The memory may include any device operable to receive, store, or recall data, including, but not limited to, electronic, magnetic, or optical memory, whether volatile or non-volatile. The memory may include code stored thereon. The code may include instructions that may be executable by the processor. The code may be created, for example, using any programming language, including but not limited to, C, C++, Java, Python, Rust, or any other programming language (including assembly languages, hardware description languages, and database programming languages). In some instances, the code may be a program that, when loaded into the processor, causes the surgical console 100 to receive and process information from one or more of subsystems 106, 110, 111, or 116 for, e.g., providing fluid control for one or more handpieces 112 or other devices in communication with the surgical console 100.

The processor may be, or include, a microprocessor, a microcontroller, an embedded microcontroller, a programmable digital signal processor, or any other programmable device operable to receive information from the memory or other devices in communication with the processor, computer 103, and/or console 100, and perform one or more operations on the received information. For example, the processor may send instructions to components of pneumatics subsystem 111 or fluidics subsystem 110, or other devices or systems in communication with computer 103 (e.g., handpieces 112), for controlling such devices and systems. The processor may also be operable to output results based on the operations performed thereby. A display screen 104 shows data provided by the processor of computer 103. In some instances, the processor may also be or include an application specific integrated circuit, a programmable gate array, programmable array logic, or any other device of combinations of devices operable to process electric signals.

Handpiece 112 may be any suitable surgical device or ophthalmic surgical instrument, e.g., a vitrectomy handpiece, other types of pneumatically-driven handpieces, and/or other suitable non-pneumatically-driven surgical handpieces. Other examples of the handpiece 112 may include an ultrasonically-driven phacoemulsification (phaco) handpiece, a laser handpiece, an irrigating cannula, a vitrectomy handpiece, or another suitable surgical handpiece.

Fluidics subsystem 110 may provide fluid control for one or more handpieces 112 (112a-c). In some embodiments, pneumatically-driven handpieces, e.g., a vitrectomy handpiece, may be operatively coupled to a surgical cassette during a surgical procedure. For example, a surgical cassette may be inserted into, attached to, and/or integrated with the fluidics subsystem 110 via a coupling mechanism. The coupling mechanism may comprise one or more of a latching mechanism, a locking mechanism, or other similar connection mechanism. When fluidics subsystem 110 is operatively coupled to a surgical cassette, fluidics subsystem 110 may control irrigation, infusion, suction, and/or aspiration of fluids through the surgical cassette and the handpiece.

Pneumatics subsystem 111 provides pneumatic power and control for one or more handpieces 112 (112a-c). For example, the handpiece 112a may be a vitrectomy handpiece driven by the pneumatics subsystem 111. The vitrectomy handpiece 112a can be actuated by pneumatic pressure in two channels, e.g., channels A and B, via a tubing set and a connector connected to a port 120, e.g., the port 120-5. When channel A is being pressurized and channel B is venting, the differential pressure between channel A and channel B may act on a diaphragm inside the vitrectomy handpiece 112a, causing the diaphragm and an attached probe cutter to move to an open position. When opposite pressurization and venting is applied to channel A and channel B, the probe cutter will move to the close position. As controlled by the pneumatics subsystem 111, movement of the probe cutter of the vitrectomy handpiece 112a in open and closed cycles can break or shear vitreous into small fragments for removal from a patient's eye.

Generally, handpiece subsystem 116 supports one or more handpieces 112. For example, handpiece subsystem 116 may manage ultrasonic oscillation for a phaco handpiece, provide laser energy to a laser handpiece, control operation of an irrigating cannula, and/or manage features of a vitrectomy handpiece. In certain embodiments, the handpieces 112 connect to the handpiece subsystem 116 via the port interface 118. For example, connectors of the handpieces 112 may be inserted into, coupled with, and/or received by at least one of the ports 120.

A connection detecting system 114 of the surgical console 100 is connected to the computer 103 and the handpiece subsystem 116. The connection detecting system 114 may determine whether the handpiece 112 is connected and/or what type of handpiece (e.g., type of surgical device) is connected to the ports 120 of surgical console 100. As such, the computer 103 can indicate, via the display screen 104, information related to the connection of the handpiece 112.

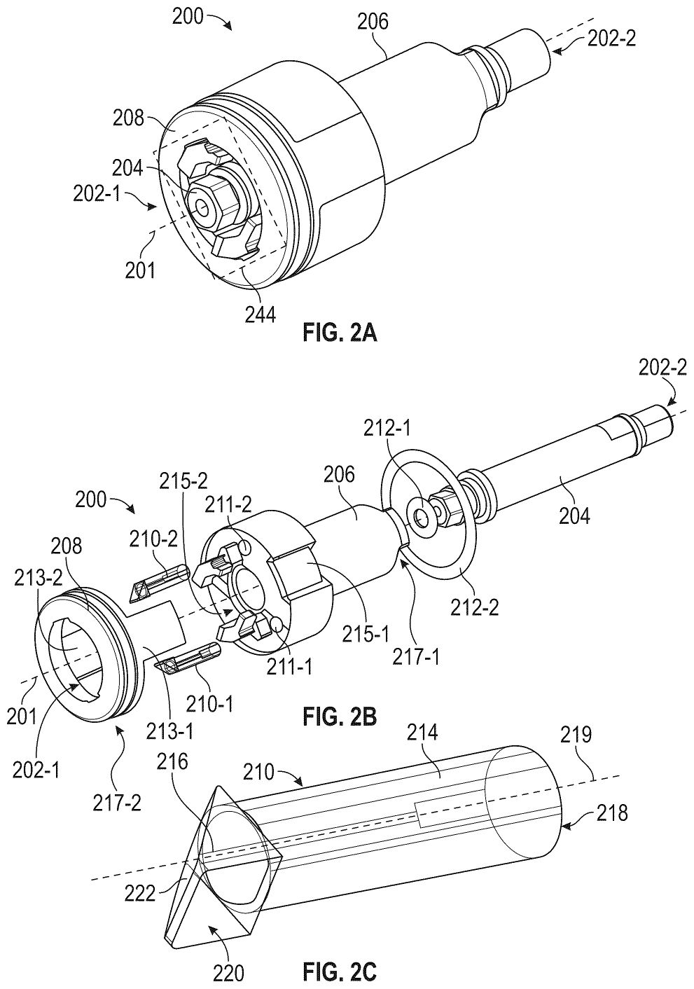

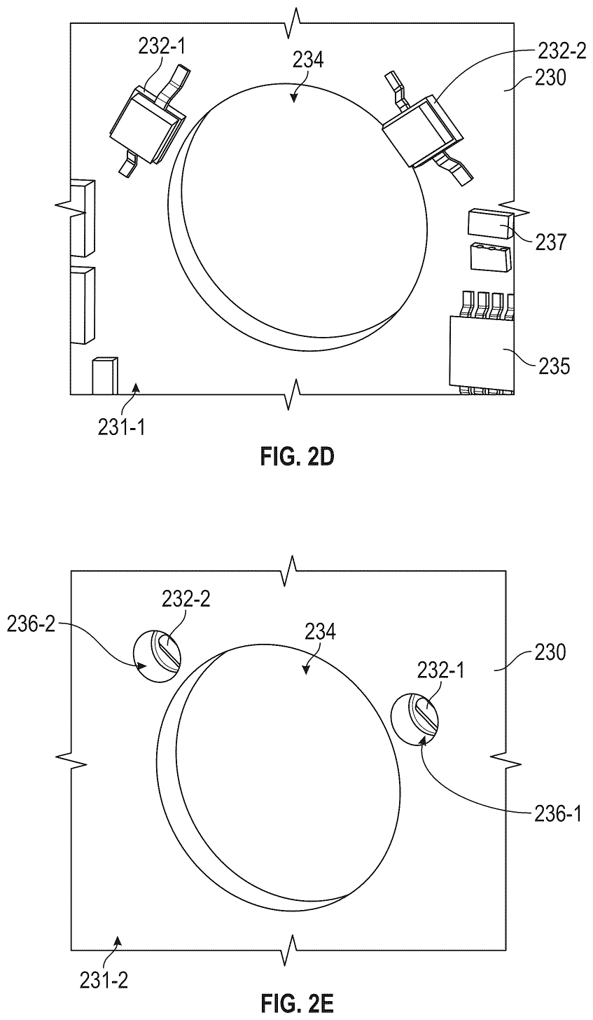

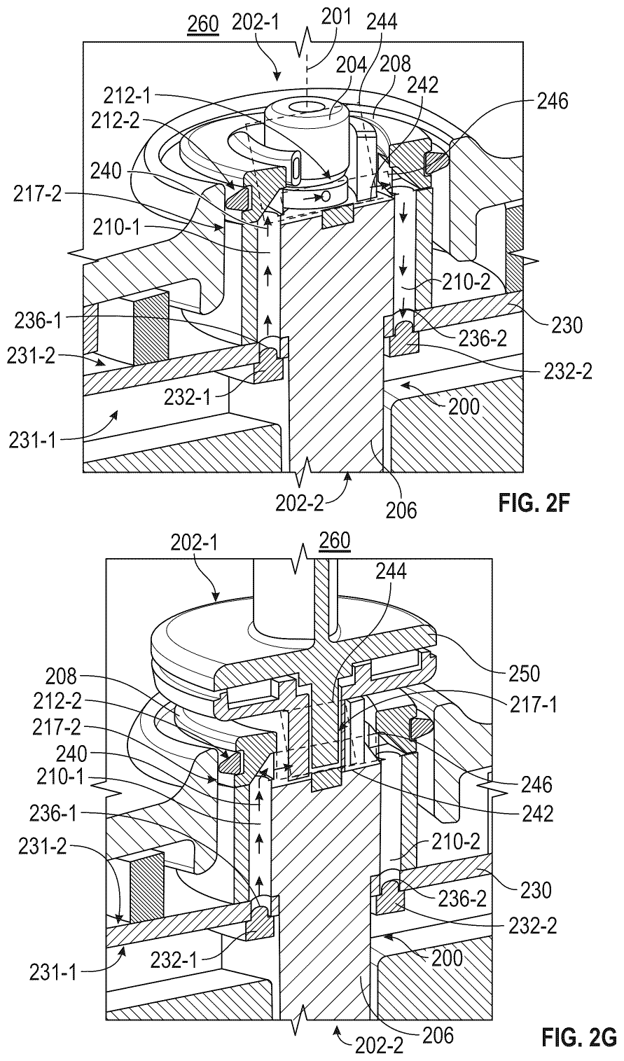

FIG. 2A is a side isometric view of an example port 200 of the ophthalmic surgical console 100 of FIG. 1A, according to certain embodiments. The port 200 may represent an embodiment of the ports 120-1, 120-2, and 120-3 of FIG. 1B. As such, the port 200 is configured to receive a connector of a surgical device, more specifically, a VFC connector, a forceps connector, a scissor connector, and/or the like. The port 200 includes a connector core 204, a body 206, a retainer cap 208, a pair of light pipes 210-1 and 210-2, and two sealing rings 212-1 and 212-2 (collectively referred to herein as “sealing rings 212”).

The connector core 204 is disposed within the body 206 along a longitudinal axis 201 of the port 200. At a first end 202-1, the connector core 204 is configured to couple with the connector of a surgical device. At a second end 202-2, the connector core 204 is configured to connect to a corresponding driver, source, or controller for the surgical device connected to the port 200 of the surgical console 100 (FIG. 1A). In certain embodiments, the connector core 204 acts as a conduit, an adapter, an interface, and/or an intermediate device which facilitates transfer of electrical signals, air, other fluids, etc., from the surgical console 100 to the surgical device. As such, the connector core 204 may comprise a conduit disposed through the connector core 204 with one or more electrical connections therein or for allowing passage of air or other fluids.

FIG. 2B is an exploded side isometric view of the example port seen in FIG. 2A, according to certain embodiments. In addition to the connector core 204, the body 206, and the retainer cap 208, the port 200 includes a pair of light pipes 210-1 and 210-2, and a pair of sealing rings 212-1 and 212-2.

The pair of light pipes 210 includes a first light pipe 210-1 and a second light pipe 210-2, which are described in further detail with reference to FIG. 2C. The first light pipe 210-1 may be disposed in a first body aperture 211-1 of the body 206, and the second light pipe 210-2 may be disposed in a second body aperture 211-2 of the body 206 (collectively referred to herein as “body apertures 211”). As such, the light pipes 210 may be embedded within, or extend through, the body 206 of the port 200 and held in position by the retainer cap 208 (best seen in FIGS. 2F and 2G). In certain embodiments, the retainer cap 208 has one or more indentations on a proximal side of the retainer cap 208 that correspond to the light pipes 210. In such embodiments, the indentations in the retainer cap 208 can control or determine the orientation of the light pipes 210. In certain embodiments, the orientation of the light pipes 210 can be controlled or determined by matching flat surfaces on the sides of the light pipes 210-1, 210-2 with flat surfaces of the body apertures 211-1, 211-2 when the light pipes 210-1, 210-2 are inserted in the body apertures 211-1, 211-2 respectively.

In certain embodiments, there may be air gaps between the light pipes 210 and the retainer cap 208. In other words, the indentations in the proximal side of the retainer cap 208 may be recessed into the retainer cap 208 such that they form air gaps between the retainer cap 208 and the light pipes 210. In certain embodiments, the light pipes 210 may be recessed away from an inner edge of the retainer cap 208 to prevent water ingress and contamination (best seen in FIGS. 2F and 2G). The retainer cap 208 is snap fit to the body 206 via tabs 213-1 and 213-2 that slip into cutouts 215-1 and 215-2, respectively, in the body 206. As such, the retainer cap 208 can be removed from the body 206, allowing the retainer cap 208 and the light pipes 210 to be serviceable and replaced without removing other parts of the port 200 when assembled.

The body apertures 211 may be located along a peripheral edge of a coupling zone 244 of the port 200 (best seen in FIG. 2F). The coupling zone 244 may be an area within the body 206 (e.g., between the connector core 204 and the body 206) that is configured to receive a connector of a surgical device.

The first sealing ring 212-1 may be disposed on a distal outer diameter (OD) surface of the connector core 204. As such, the first sealing ring 212-1 is configured to form a seal between the connector core 204 and an inside diameter (ID) surface 217-1 of the connector 250 (best seen in FIGS. 2F-2G) when the connector 250 is connected to the port 200. The second sealing ring 212-2 may be disposed between an outer diameter (OD) surface 217-2 of the retainer cap 208 and an inside diameter (ID) surface of the external face 122 of the surgical console 100 (FIG. 1A). As such, the second sealing ring 212-2 is configured to form a seal between the retainer cap 208 and the external face 122 of the surgical console 100. As an example, the sealing rings 212 are O-rings formed of thermoelastomeric materials, rubbers, other elastomeric materials, and/or the like.

Although shown in a particular arrangement, the body apertures 211, the light pipes 210, and the sealing rings 212 may have a different arrangement in certain embodiments.

FIG. 2C is a side isometric view of a light pipe 210 seen in FIG. 2B, according to certain embodiments. That is, the light pipe 210 seen in FIG. 2C may be representative of the first light pipe 210-1 and/or the second light pipe 210-2 seen in FIG. 2B. In certain embodiments, the light pipe 210 includes a solid mass made of a transparent material such as, e.g., an acrylic or polycarbonate material fabricated by machining or injection molding. Some or all surfaces of the light pipe 210 may be smooth, such that the surfaces are configured to allow light to be transmitted therethrough, or to allow total internal reflection. A surface geometry and material type of the light pipe 210 may enable the total internal reflection, such that the light can be transmitted through the light pipe 210 and change its direction at an outcoupling surface (i.e., exit point, e.g., end face 220)) of the light pipe 210 from its original direction at an incoupling surface (i.e., entry point, e.g., end face 218) of the light pipe 210 with minimum loss.

The light pipe 210 includes a body 214 and an angled end 216 connected thereto. The body 214 may generally include an elongated body or other similar cross-sectional profile for transmitting light along a longitudinal axis 219 of the light pipe 210. The angled end 216 may have flat surfaces for turning, or directing, the light in a direction orthogonal to the longitudinal axis 219.

The body 214 is shown as having a partially cylindrical shape, but may also be rectangular, circular, or another similar shape. As an example, the body may have a length between 8 millimeters (mm) and 10 mm (e.g., between 8.2 mm and 9.8 mm, 8.4 mm and 9.6 mm, and 8.6 mm and 9.4 mm), and a width between 2 mm and 3 mm (e.g., between 2.2 mm and 2.8 mm or 2.4 mm and 2.6 mm).

The body 214 includes a first end face 218 at one end of the body 214. The first end face 218 may be configured to be disposed adjacent to, or couple with, one of two apertures (light apertures 236-1 and/or 236-2) in a printed circuit board (PCB) (PCB 230), through which light may be propagated to or from a detector (detector 232-2) or an emitter (emitter 232-1), respectively. As such, the first end face 218 can be configured to receive light being distally emitted or propagated through one of the apertures of the PCB, such that the light can then be propagated through the body 214 of the light pipe 210, or vice versa. In other words, the first end face 218 may allow light that is generated by the emitter and propagated through one of the PCB apertures to enter the light pipe 210 (when paired with the emitter), or the first end face 218 may allow light to exit the light pipe 210 through the other of the apertures toward a detector for detection (when paired with a detector).

The first end face 218 is generally orientated perpendicular to the longitudinal axis 219 of body 214. The first end face 218 may be planar or non-planar, e.g., a convex surface or a concave surface. As such, the first end face 218 may be configured to collimate the light in the light pipe 210, enhance flux coupling with the emitter, or better focus the light onto the detector. In some embodiments, instead of a non-planar surface of the first end face 218, one or more lenses may be placed in front of the first end face 218 to collimate the light, enhance the flux coupling with the emitter, or better focus the light onto the detector.

The angled end 216 of the light pipe 210 is disposed at an end of the body 214 opposite of the first end face 218. The angled end 216 includes a second end face 220 and an angled surface 222. The second end face 220 may be configured to be disposed adjacent to, or couple with, a detection zone of the port 200 (best seen in FIG. 2F). As such, the second end face 220 is configured to allow light to pass between the light pipe 210 and the detection zone. The second end face 220 may be planar or non-planar. For example, the second end face 220 may be a convex surface for collimating light exiting and/or entering the light pipe 210.

In certain embodiments, the second end face 220 is disposed substantially orthogonal to the first end face 218 and the angled surface 222 is disposed at about 45 degrees relative to the first end face 218 (in such embodiments, the angles of the second end face 220 and the angled surface 222 also be slightly more or less (e.g., ±1 degree) than 90 degrees or 45 degrees, respectively, relative to the first end face 218). The angled surface 222, when disposed at 45 degrees relative to the first end face 218, and the second end face 220, when disposed at 90 degrees relative to the first end face 218, cause light propagated through the light pipe 210 to change direction by 90 degrees between entering the first end face 218 and exiting the second end face 220, or vice versa. As such, when light is emitted onto the angled surface 222, the light may be redirected towards the first end face 218 or the second end face 220, depending on the light's original direction. In some embodiments, the light changes direction by about 90 degrees with total internal reflection occurring at side wall surfaces of the body 214 and the angled surface 222.

Although the angled surface 222 and the second end face 220 are described as being disposed about 45 degrees and 90 degrees relative to the first end face 218, respectively, the angled surface 222 may be more or less than 45 degrees and the second end face 220 may be more or less than 90 degrees relative to the first end face 218. In other words, the angled surface 222 and the second end face 220 may be angled relative to the first end face 218 so long as total internal reflection occurs at the angled surface 222 and the light pipe 210 directs light appropriately.

FIGS. 2D and 2E are opposing side isometric views of an example PCB 230 and are described together herein for clarity purposes. The PCB 230 is configured to mechanically and electrically support and/or interconnect various electronic devices of the connection detecting system, such as an emitter 232-1 and a detector 232-2. FIG. 2D illustrates a first side 231-1 of the PCB 230, and FIG. 2E illustrates a second side 231-2 of the PCB 230. The emitter 232-1 and the detector 232-2 are mounted (e.g., reverse mounted) to the first side 231-1 of the PCB 230. The emitter 232-1 is configured to generate a light for propagation through a light transmission system 240 (best seen in FIGS. 2F and 2G), and the detector 232-2 is configured to detect the light propagated through the light transmission system 240.

The PCB 230 includes a port aperture 234 and a pair of light apertures: a first light aperture 236-1 for the emitter 232-1, and a second light aperture 236-2 for the detector 232-2 (collectively referred to herein as “light apertures 236”). The PCB 230 may also include one or more other electrical components. For example, the PCB 230 may include a driver that is connected to the emitter 232-1 and/or the detector 232-2. The function of the driver is described in further detail with reference to FIGS. 2F and 2G.

The port 200 may be disposed through the port aperture 234 (best seen in FIGS. 2F and 2G). The port aperture 234 may have a diameter between 5 mm and 15 mm (e.g., between 6 mm and 14 mm, 7 mm and 13 mm, and 8 mm and 12 mm). Although the port aperture 234 is shown as having a circular shape, the port aperture may have a D-like shape, a cylindrical shape, a rectangular shape, or other shape corresponding to a shape of a port disposed therethrough.

In certain embodiments, the emitter 232-1 includes a light source configured to generate light, and the detector 232-2 includes a sensor configured to detect the light generated by the light source. The controller 235 may be connected to and communicate with the emitter 232-1 and the detector 232-2 via wiring through or on the PCB 230. As such the controller 235 may be configured to control the emitter 232-1 and communicate with the detector 232-2 to determine whether light is detected.

In certain embodiments, the emitter 232-1 includes a light-emitting diode (LED) emitter such as, e.g., an infrared (IR) emitting diode with a peak wavelength of 940 nanometer (nm). In some embodiments, the emitter 232-1 is a narrow beam type LED (e.g., ±7° angle of half intensity). The detector 232-2 may be a photodiode or a phototransistor configured to detect the light emitted by the emitter 232-1. As an example, the detector 232-2 may be a photodiode with a daylight blocking filter that is configured to detect IR light from the emitter 232-1 having wavelengths between 830 nm and 950 nm. In some embodiments, the detector 232-2 is a wider angle photodiode or a wider angle phototransistor (e.g., ±15° angle of half intensity). In certain embodiments, the emitter 232-1 may be replaced with another type of light or radiation source configured to be passed through the light transmission system 240 and detected by another corresponding type of light or radiation detector 232-2 to determine a device connection.

The emitter 232-1 and the detector 232-2 are mounted to the first side 231-1 of the PCB 230. Light generated and/or detected by the emitter 232-1 and the detector 232-2 passes through the PCB 230, for example, through the pair of light apertures 236. As such, the emitter 232-1 is aligned with the first light aperture 236-1, and the detector 232-2 is aligned with the second light aperture 236-2. Correspondingly, the pair of light pipes 210-1 and 210-2 shown in FIG. 2B are disposed against the second side 231-2 of the PCB 230 (best seen in FIGS. 2F and 2G), such that the light pipes 210-1 and 210-2 are axially aligned with the emitter 232-1 and the detector 232-2, respectively. Although the emitter 232-1, the detector 232-2, and the light apertures 236 are disposed along a peripheral edge of the port aperture 234 (i.e., not aligned with a center of the port aperture 234), the emitter 232-1, the detector 232-2, and the light apertures 236 may be disposed at opposing ends of the port aperture 234 (i.e., aligned with the center of the port aperture 234), or in another similar arrangement.

The PCB 230 may also include one or more circuits of an RFID reader 237, which may include an antenna coil near the port aperture 234. The controller 235 may be connected to the RFID reader 237 via wiring through or on the PCB 230. The RFID reader 237 may be configured to detect and read an RFID tag of a surgical device when the surgical device is connected to the port 200 (best seen in FIG. 2G) or placed in close proximity thereto. Using information captured by the RFID reader 237, the controller 235 may be configured to determine whether and what surgical device is connected to the port 200. The RFID reader 237 may be implemented with the emitter 232-1 and the detector 232-2 to enhance reliability when determining whether a surgical device is properly connected.

In certain embodiments, the RFID reader 237 may stay on continuously (e.g., through the controller 235). If the detector 232-2 detects light emitted by the emitter 232-1, then the connection detecting system determines that the surgical device is not properly connected (or that nothing is connected) to the port 200. In other words, regardless of whether the RFID reader 237 detects and/or reads an RFID tag of the connector of the surgical device, if the detector 232-2 detects light emitted by the emitter 232-1, the system determines that the surgical device is not properly connected to the port. If the detector 232-2 does not detect light, then the connection detecting system determines that the surgical device is properly connected to the port 200 once the RFID reader 237 detects and reads the RFID tag. However, if device identification information does not match the port 200, then the system may determine that an incorrect device is connected.

In certain embodiments, the RFID reader 237 may not stay on continuously. If the detector 232-2 detects light emitted by the emitter 232-1, then the connection detecting system determines that the surgical device is not properly connected (or that nothing is connected) to the port 200, and the RFID reader 237 is not turned on. If the detector 232-2 does not detect light, then the controller 235 turns on the RFID reader 237 to determine whether a correct surgical device is properly connected to the port 200. That is, the RFID reader 237 detects and reads the RFID tag in the connector of the surgical device to determine whether the device identification information matches the port 200. If the device identification information matches, and the detector 232-2 does not detect light, then it is determined that the surgical device is properly connected. However, if device identification information does not match the port 200, then it is determined that an incorrect device is connected. Subsequently, the controller 235 may turn off the RFID reader 237 until a next cycle of detector 232-2 changes state from detecting light to not detecting light.

FIG. 2F is a cross-sectional isometric view of an example connection detecting system 260 configured to connect with a surgical device, according to certain embodiments. The connection detecting system 260 is operably coupled with the port 200, the emitter 232-1, the detector 232-2, and the controller 235, as described with reference to FIGS. 2A-2E. In FIG. 2F, the port 200 is disposed through the external face 122 of the surgical console 100 (FIGS. 1A and 1B), and through the port aperture 234 of the PCB 230. As such, the first end 202-1 of the port 200 may be accessible from outside the surgical console 100, and the second end 202-2 may be accessible from inside the surgical console 100 and/or coupled to other components or systems within the surgical console 100.

The port 200 includes a base surface 242 and a coupling zone 244 near the first end 202-1. The base surface 242 surrounds the connector core 204 and may engage with a connector of a surgical device (e.g., be disposed against the connector). The coupling zone 244 may include an area between the connector core 204 and the retainer cap 208 through which the connector of the surgical device is configured to be disposed when the surgical device is coupled to the surgical console 100.

In certain embodiments, the port 200 includes a light transmission system 240 through which light may be propagated. The light transmission system 240 includes the pair of light pipes 210 and a detection zone 246 through which the connector of the surgical device is configured to be disposed when the surgical device is coupled to the surgical console. In certain embodiments, the detection zone 246 is disposed across at least a portion of the coupling zone 244 (e.g., disposed along a peripheral edge of the coupling zone 244.

The emitter 232-1 is configured to generate a light for propagation through the light transmission system 240, and the detector 232-2 is configured to detect the light propagated through the light transmission system 240. For example, the emitter 232-1 is configured to generate the light which may be propagated through the first light aperture 236-1 of the PCB 230, the first light pipe 210-1, the detection zone 246, the second light pipe 210-2, and the second light aperture 236-2. The detector 232-2 is configured to detect the light generated by the emitter 232-1 and propagated through the light transmission system 240.

Since the emitter 232-1 is configured to generate the light propagated through the light transmission system 240, a first end face of the first light pipe 210-1 is disposed against the PCB 230, and a second end face of the first light pipe 210-1 is disposed above the base surface 242 of the coupling zone 244. Light is generated by the emitter 232-1 and transmitted through the first light pipe 210-1, which then propagates across the coupling zone 244 to the second light pipe 210-2. Similarly, a second end face of the second light pipe 210-2 is disposed above the base surface 242 of the coupling zone 244, and a first end face of the second light pipe 210-2 is disposed against the PCB 230. The longitudinal axis 219 of the light pipes 210 may therefore be parallel relative to the longitudinal axis 201 of the port 200. In this manner, the light is propagated through the light pipes 210, and across (and above) the base surface 242 of the coupling zone 244 between the light pipes 210. The light propagated across (and above) the base surface 242 between the light pipes 210 forms the detection zone 246.

In certain embodiments, the controller 235, connected to the emitter 232-1 and the detector 232-2, is configured to determine whether the surgical device is connected to the port 200 based on whether light is detected by the detector 232-2. As an example, when the connector of the surgical device is improperly connected or not connected, light propagates through the light transmission system 240 and passes through the detection zone 246 as shown in FIG. 2F, and the detector 232-2 detects the light. When the detector 232-2 detects the light, this indicates that the connector is improperly connected or not connected because the connector is not completely engaged with the base surface 242 and/or completely disposed through the coupling zone 244 and/or the detection zone 246. In other words, the connector of the surgical device is improperly connected or may not be connected if the connector is not contacting, abutting, engaging, or pressing against the base surface 242.

FIG. 2G is a cross-sectional isometric view of the example connection detecting system 260 of FIG. 2F when properly connected with a connector 250 of a surgical device, according to certain embodiments. The connector 250 of the surgical device is disposed between the connector core 204 and the retainer cap 208, against the body 206. In other words, the connector 250 is disposed over the connector core 204, such that the connector 250 is disposed in the coupling zone 244 against the base surface 242. The connector 250 is therefore also disposed in the detection zone 246.

Because the connector 250 occupies the detection zone 246, the connector 250 blocks or obstructs the propagation of light through the light transmission system 240. In particular, the connector 250 obstructs the detection zone 246 and prevents the propagation of light from one light pipe to the other light pipe (i.e., after the light is redirected by the first light pipe 210-1). Since the light cannot pass through the detection zone 246, the detector 232-2 does not detect the light propagated through the light transmission system 240. When the detector 232-2 does not detect the light, this indicates that the connector 250 is properly connected and completely engaged with the base surface 242, and/or completely disposed through the coupling zone 244 and/or the detection zone 246. In other words, the connector 250 of the surgical device is properly connected if the connector 250 is contacting, abutting, engaging, or pressing against the base surface 242.

The connector 250 of the surgical device may also include an RFID tag configured to be detected and read by the RFID reader 237 of the connection detecting system 260. The controller 235 of the connection detecting system 260 may be connected to the RFID reader 237 and may be configured to determine whether the surgical device is connected to the port 200 based on whether the RFID tag is detected and read.

In certain embodiments, the light pipes 210 can be combined or integrated into a single light pipe or component. That is, the light pipes 210 may be integrated with the body 206 of the port 200. As such, there may be less reliance on components of the port 200 (e.g., the body 206 and/or the retainer cap 208) for orienting the light pipes 210.

In certain embodiments, the connection detecting system 260 does not include light pipes 210, and instead, internal surfaces of the body apertures 211-1, 211-2 and/or angled surfaces inside the retainer cap 208 may be coated with a reflective coating configured to propagate light. That is, rather than utilizing light pipes 210, the reflective coating can be used to propagate light through light transmission system 240 from the emitter 232-1 to the detector 232-2 in a manner similar to when the light pipes 210 are used.

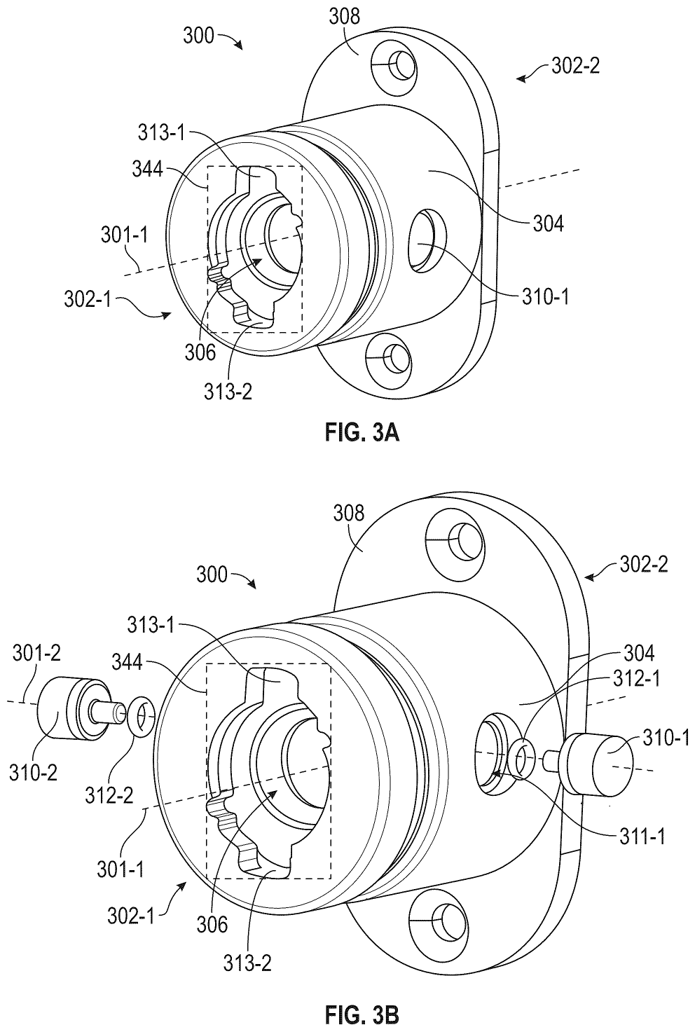

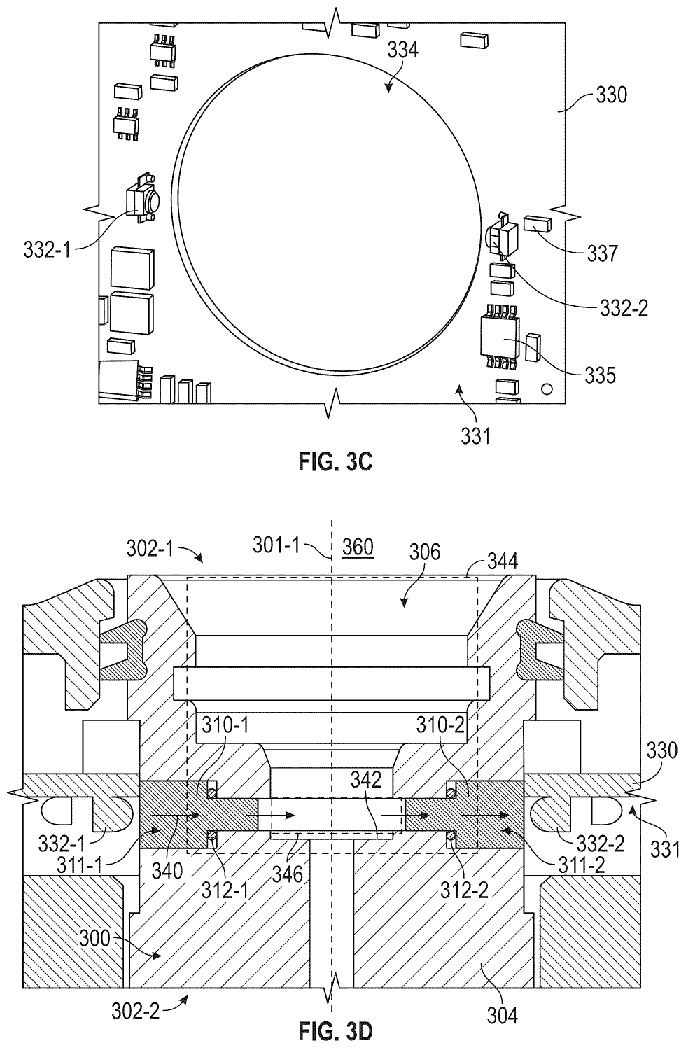

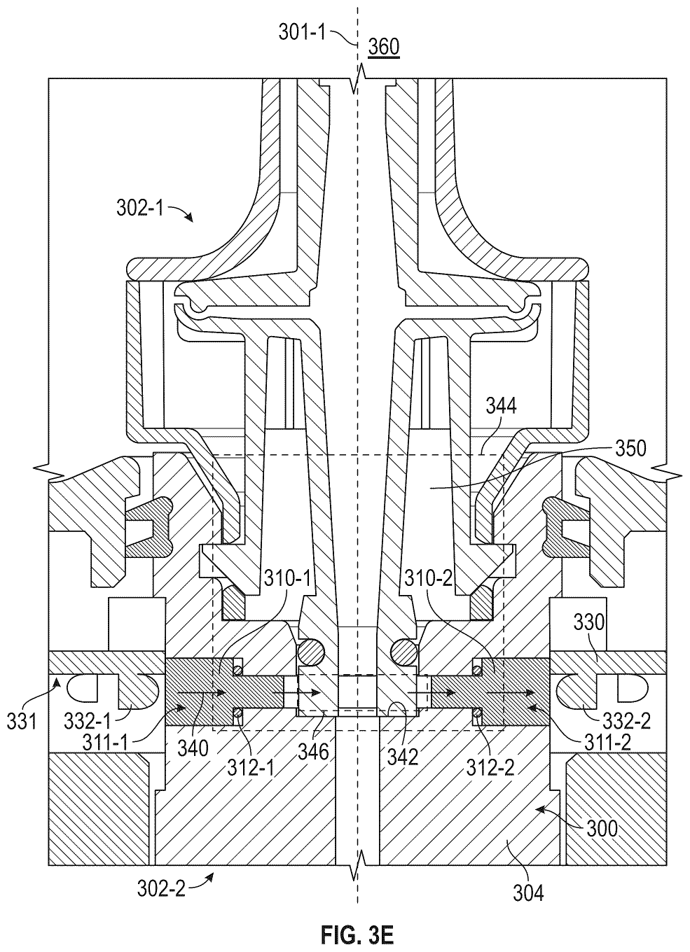

FIG. 3A is a side isometric view of another example port 300 of the ophthalmic surgical console 100 (FIG. 1A), according to certain embodiments. The port 300 may represent an embodiment of the port 120-4 of FIG. 1B. As such, the port 300 is configured to receive a connector of a surgical device, more specifically, a low pressure air source (LPAS) connector. The port 300 includes a body 304 with an external connector opening 306 and a base 308, and a plurality of light pipes: a first light pipe 310-1 and a second light pipe 310-2 (collectively referred to herein as “light pipes 310”) (best seen in FIG. 3B).

The external connector opening 306 is at a first end 302-1 of the port 300. The connector of a surgical device may connect to the port 300 by being inserted into the external connector opening 306 (best seen in FIG. 3E). As an example, the external connector opening 306 may have a shape corresponding to the connector of the surgical device.

The external connector opening 306 includes two cutouts: a first cutout 313-1 and a second cutout 313-2 (collectively referred to herein as “cutouts 313”). The cutouts 313 facilitate proper alignment and locking with the connector 350 of the surgical device. That is, the connector 350 is inputted into the external connector opening 306 and tabs of the connector 350 fit through the cutouts 313, after which the connector is rotated such that the tabs are then locked in place.

The base 308 is at a second end 302-2 of the port 300. The base 308 may be mounted within the surgical console 100 (FIG. 1A), such that a corresponding driver, source, or controller for a surgical device may connect to the base 308. The base 308 may be connected to the external connector opening 306 along a longitudinal axis 301-1 of the port 300.

The light pipes 310 are disposed along a center portion of the body 304, outside the external connector opening 306. In other words, the light pipes 310 may be on opposite sides of the external connector opening 306 and aligned along a lateral axis 301-2. The lateral axis 301-2 may be orthogonal to the longitudinal axis 301-1. The area within the external connector opening 306 between the light pipes 310 may be considered a detection zone 346 (best seen in FIGS. 3D and 3E).

Similar to the light pipes 210, light pipes 310 may include solid bodies made of a transparent material such as, e.g., an acrylic or a polycarbonate made by machining or injection molding. In certain embodiments, some or all surfaces of the light pipes 310 are smooth and configured to facilitate light transmission therethrough, or to enable total internal reflection. A surface geometry and a material type of the light pipes 310 are configured to enable total internal reflection, such that light can be transmitted through the light pipes 310 with minimum loss.

FIG. 3B is an exploded side isometric view of the example port 300 seen in FIG. 3A, according to certain embodiments. In addition to the body 304, the external connector opening 306, the base 308, and the light pipes 310, the port 300 includes a pair of sealing rings: a first sealing ring 312-1 and a second sealing ring 312-2 (collectively referred to herein as “sealing rings 312”). As an example, the sealing rings 312 are O-rings.

The first sealing ring 312-1 may be disposed between the body 304 and the first light pipe 310-1. As such, the first sealing ring 312-1 is configured to form a seal between the body 304 and the first light pipe 310-1. The second sealing ring 312-2 may be disposed between the body 304 and the second light pipe 310-2. As such, the second sealing ring 312-2 is configured to form a seal between the body 304 and the second light pipe 310-2. As an example, the sealing rings 312 are O-rings formed of thermoelastomeric materials, rubbers, other elastomeric materials, and/or the like.

The first light pipe 310-1 and the first sealing ring 312-1 may be disposed in a first body aperture 311-1, and the second light pipe 310-2 and the second sealing ring 312-2 may be disposed in a second body aperture 311-2 (collectively referred to herein as “body apertures 311”) (best seen in FIG. 3D). That is, the light pipes 310 and the sealing rings 312 are press fitted in the body apertures 311 of the body 304, which seals against pneumatic pressure of the port 300. The body apertures 311 may be located along a center of a coupling zone of the port 300 (best seen in FIG. 3D). Although shown in a certain arrangement, the body apertures 311, the light pipes 310, and the sealing rings 312 may have a similar or different arrangement (e.g., around the body 304).

FIG. 3C is a first side 331 isometric view of another example PCB 330, according to certain embodiments. The PCB 330 is configured to mechanically and electrically support and/or interconnect various electronic devices of the connection detecting system, such as an emitter 332-1 and a detector 332-2. The PCB 330 includes the emitter 332-1, the detector 332-2, and a port aperture 334 as described with reference to FIG. 2D.

As opposed to the emitter 232-1 and the detector 232-2 shown in FIG. 2D, the emitter 332-1 and the detector 332-2 are mounted across a center of the port aperture 334, orthogonal to the PCB 330. As such, when the port 300 is disposed through the port aperture 334, the emitter 332-1 and the detector 332-2 align with the light pipes 310 and emit and/or detect light therethrough. In certain embodiments, the emitter 332-1 and the detector 332-2 may be the same as the emitter 232-1 and the detector 232-2, respectively. In certain embodiments, the emitter 332-1 may be replaced with another type of light or radiation source configured to be passed through the light transmission system 340 (best seen in FIGS. 3D and 3E) and detected by another type of detector 332-2 configured to detect a corresponding light or radiation detecting source to determine a device connection.

In certain embodiments, the emitter 332-1 and the detector 332-2 are connected to and communicate with a controller 335. For example, the controller 335 may be connected to the emitter 332-1 and/or the detector 332-2 via wiring through or on the PCB 330. The function of the controller 335 is described in further detail with reference to FIGS. 3D and 3E.

The PCB 330 may also include one or more circuits of an RFID reader 337, which may include an antenna coil near the port aperture 334. The controller 335 may be connected to the RFID reader 337 via wiring through or on the PCB 330. The RFID reader 337 may be configured to detect and read an RFID tag of a surgical device when the surgical device is connected to the port 300 (best seen in FIG. 3E) or placed in close proximity thereto. Using information captured by the RFID reader 337, the controller 335 may be configured to determine whether and what surgical device is connected to the port 300. The RFID reader 337 may be implemented with the emitter 332-1 and the detector 332-2 to enhance reliability when determining whether a surgical device is properly connected.

In certain embodiments, the RFID reader 337 may stay on continuously (e.g., through the controller 335). If the detector 332-2 detects light emitted by the emitter 332-1, then the connection detecting system determines that the surgical device is not properly connected (or that nothing is connected) to the port 300. In other words, regardless of whether the RFID reader 337 detects and/or reads an RFID tag of the connector of the surgical device, if the detector 332-2 detects light emitted by the emitter 332-1, the system determines that the surgical device is not properly connected to the port. If the detector 332-2 does not detect light, then the connection detecting system determines that the surgical device is properly connected to the port 300 once the RFID reader 337 detects and reads the RFID tag. However, if device identification information does not match the port 300, then the system may determine that an incorrect device is connected.

In certain embodiments, the RFID reader 337 may not stay on continuously. If the detector 332-2 detects light emitted by the emitter 332-1, then the connection detecting system determines that the surgical device is not properly connected (or that nothing is connected) to the port 300, and the RFID reader 337 is not turned on. If the detector 332-2 does not detect light, then the controller 335 turns on the RFID reader 337 to determine whether a correct surgical device is properly connected to the port 300. That is, the RFID reader 337 detects and reads the RFID tag in the connector of the surgical device to determine whether the device identification information matches the port 300. If the device identification information matches, and the detector 332-2 does not detect light, then it is determined that the surgical device is properly connected. However, if device identification information does not match the port 300, then it is determined that an incorrect device is connected. Subsequently, the controller 335 may turn off the RFID reader 337 until a next cycle of detector 332-2 changes state from detecting light to not detecting light.

The port 300 may be disposed through the port aperture 334 (best seen in FIGS. 3D and 3E). The port aperture 334 may have a diameter between 25 mm and 30 mm (e.g., between 25.5 mm and 29.5 mm, 26 mm and 29 mm, and 26.5 mm and 28.5 mm). Although the port aperture 334 is shown as having a circular shape, the port aperture may have D-like shape, a cylindrical shape, a rectangular shape, or other shape corresponding to a shape of a port disposed therethrough.

FIG. 3D is a cross-sectional isometric view of another example connection detecting 360 system configured to connect with a surgical device, according to certain embodiments. The connection detecting system 360 is operably coupled with the port 300, the emitter 332-1, the detector 332-2, and the controller 335, as described with reference to FIGS. 3A-3C. In FIG. 3D, the port 300 is disposed through the external face 122 of the surgical console 100 (FIGS. 1A and 1B), and through the port aperture 334 of the PCB 330. As such, the first end 302-1 of the port 300 may be accessible from outside the surgical console 100, and the second end 302-2 may be accessible from inside the surgical console 100 and/or coupled to other components or systems within the surgical console 100.

The port 300 includes a base surface 342 and a coupling zone 344 near the first end 302-1. The base surface 342 surrounds the body 304 and may engage with a connector of a surgical device (e.g., be disposed against the connector). The coupling zone 344 may include an area of the external connector opening 306 within the body 304 through which the connector of the surgical device is configured to be disposed when the surgical device is coupled to the surgical console 100.

In certain embodiments, the port 300 includes a light transmission system 340 through which light may be propagated. The light transmission system 340 includes the pair of light pipes 310 and a detection zone 346 through which the connector of the surgical device is configured to be disposed when the surgical device is coupled to the surgical console. In certain embodiments, the detection zone 346 is disposed across the coupling zone 344 (e.g., disposed across a center of the coupling zone 344).

The emitter 332-1 is configured to generate a light for propagation through the light transmission system 340, and the detector 332-2 is configured to detect the light propagated through the light transmission system 340. For example, the emitter 332-1 is configured to generate the light which may be propagated through the first light pipe 310-1, the detection zone 346, and the second light pipe 310-2. The detector 332-2 is configured to detect the light generated by the emitter 332-1 and propagated through the light transmission system 340.

Since the emitter 332-1 is configured to generate the light propagated through the light transmission system 340, end faces of the light pipes 310 may be disposed against the emitter 332-1 and the detector 332-2, and inside end faces of the light pipes 310 may be disposed above the base surface 342 of the coupling zone 344. Light is generated by the emitter 332-1 and transmitted through the first light pipe 310-1, which then propagates across the coupling zone 344 to the second light pipe 310-2. A longitudinal axis of the light pipes 310 may therefore be orthogonal to the longitudinal axis 301-1 of the port 300. In this manner, the light is propagated through the light pipes 310, and across (and above) the base surface 342 of the coupling zone 344 between the light pipes 310. The light propagated across (and above) the base surface 342 between the light pipes 310 forms the detection zone 346.

In certain embodiments, the controller 335, connected to the emitter 332-1 and the detector 332-2, is configured to determine whether the surgical device is connected to the port 300 based on whether the light is detected by the detector 332-2. As an example, when the connector of the surgical device is improperly connected or not connected, light propagates through the light transmission system 340 and passes through the detection zone 346 as shown in FIG. 3D. When the detector 332-2 detects the light, this indicates that the connector is improperly connected or not connected because the connector is not completely engaged with the base surface 342 and/or completely disposed through the coupling zone 344 and/or the detection zone 346. In other words, the connector of the surgical device is improperly connected or may not be connected if the connector is not contacting, abutting, engaging, or pressing against the base surface 342.

FIG. 3E is a cross-sectional isometric view of the example connection detecting system 360 of FIG. 3D when properly connected with a connector 350 of a surgical device, according to certain embodiments. The connector 350 of the surgical device is disposed through the external connector opening 306 against the body 304. In other words, the connector 350 is disposed in the external connector opening 306, such that the connector 350 is disposed in the coupling zone 344 against the base surface 342. The connector 350 is therefore also disposed in the detection zone 346.

Because the connector 350 occupies the detection zone 346, the connector 350 blocks or obstructs the propagation of light through the light transmission system 340. In particular, the connector 350 obstructs the detection zone 346 and prevents the propagation of light from one light pipe to the other light pipe (i.e., after the light is propagated through the first light pipe 310-1). Since the light cannot pass through the detection zone 346, the detector 332-2 does not detect the light propagated through the light transmission system 340. When the detector 332-2 does not detect the light, this indicates that the connector 350 is properly connected and completely engaged with the base surface 342, and/or completely disposed through the coupling zone 344 and/or the detection zone 346. In other words, the connector 350 of the surgical device is properly connected if the connector 350 is contacting, abutting, engaging, or pressing against the base surface 342.

The connector 350 of the surgical device may also include an RFID tag configured to be detected and read by the RFID reader 337 of the connection detecting system 360. The controller 335 of the connection detecting system 360 may be connected to the RFID reader 337 and may be configured to determine whether the surgical device is connected to the port 300 based on whether the RFID tag is detected and read.

Further, although the port 300 is shown and described as being configured to receive an LPAS connector, a similar port and light pipe configuration may also be implemented in a port configured to receive a vitrectomy probe connector, a VFC connector, a forceps connector, a scissors connector, or an illumination probe connector. In certain embodiments, the external connector opening 306 may be configured to receive connectors of other surgical devices, such that the external connector opening 306 may have a different shape and/or dimensions that correspond with the connectors of other surgical devices.

The present disclosure may be embodied in other specific forms without departing from its spirit or essential characteristics. The described embodiments are to be considered in all respects only as illustrative and not restrictive. The scope of the present disclosure is, therefore, indicated by the appended Claims rather than by this Detailed Description. All changes which come within the meaning and range of equivalency of the Claims are to be embraced within their scope.

Reference throughout this specification to features, advantages, or similar language does not imply that all of the features and advantages that may be realized with the present disclosure should be or are in any single embodiment of the disclosure. Rather, language referring to the features and advantages is understood to mean that a specific feature, advantage, or characteristic described in connection with an embodiment is included in at least one embodiment of the present disclosure. Thus, discussions of the features and advantages, and similar language, throughout this specification may, but do not necessarily, refer to the same embodiment.

Furthermore, the described features, advantages, and characteristics of the disclosure may be combined in any suitable manner in one or more embodiments. One skilled in the relevant art will recognize, in light of the description herein, that the disclosure can be practiced without one or more of the specific features or advantages of a particular embodiment. In other instances, additional features and advantages may be recognized in certain embodiments that may not be present in all embodiments of the present disclosure.

Reference throughout this specification to “one embodiment”, “an embodiment”, or similar language means that a particular feature, structure, or characteristic described in connection with the indicated embodiment is included in at least one embodiment of the present disclosure. Thus, the phrases “in one embodiment”, “in an embodiment”, and similar language throughout this specification may, but do not necessarily, all refer to the same embodiment.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

### EXAMPLE EMBODIMENTS

Embodiment 1: A connection detecting system for a surgical console, the surgical console comprising: a port for receiving a connector of a surgical device, the port comprising: a port body with apertures; a light transmission system comprising: a detection zone through which the connector of the surgical device is configured to be disposed when the surgical device is coupled to the surgical console; and a pair of light pipes disposed in the apertures of the port body at opposite ends of the detection zone, wherein a longitudinal axis of each of the light pipes is parallel relative to a longitudinal axis of the port; and a retainer cap configured to hold the pair of light pipes in place; and an emitter configured to generate a light for propagation through the light transmission system; and a detector configured to detect the light propagated through the light transmission system; and a controller, connected to the emitter and the detector, configured to determine whether the surgical device is connected to the port based on whether the light is detected by the detector.

Embodiment 2: The connection detecting system of Embodiment 1, wherein each of the light pipes comprises: an elongated cylindrical or similar cross section profile body with a first end face perpendicular or near perpendicular to a longitudinal axis of the body; and an angled end with a second end face parallel or near parallel to the longitudinal axis of the body and an angled surface 45 degrees or close to 45 degrees relative to the longitudinal axis of the body, the second end face configured to redirect the light propagated thereon.

Embodiment 3: The connection detecting system of Embodiment 2, wherein each of the pair of light pipes comprises: smooth surfaces; and a shape and a surface geometry configured to enable total internal reflection, which transmits the light through the light pipe and turns its direction 90 degrees as the light enters the light pipe through the first end face and exits out the second end face or vice versa.

Embodiment 4: The connection detecting system of Embodiment 2, wherein the first end face and the second end face are planar surfaces.

Embodiment 5: The connection detecting system of Embodiment 2, wherein the first end face and the second end face are non-planar surfaces.

Embodiment 6: The connection detecting system of Embodiment 5, wherein the non-planar surfaces are convex surfaces or concave surfaces.

Embodiment 7: The connection detecting system of Embodiment 1, wherein the apertures of the port body have cross section profiles matching a cross section profile of the light pipe body, and wherein the cross section profiles of the apertures control an orientation of the pair of light pipes such that the pair of light pipes are aligned at second end faces of each of the pair of light pipes.

Embodiment 8: The connection detecting system of Embodiment 1, wherein recess cutout surfaces of the retainer cap match side walls of an angled end and a top edge of each of the pair of light pipes, the recess cutout surfaces configured to: control an orientation of each of the pair of light pipes such that the pair of light pipes are aligned at a second end face; and hold a position of the pair of light pipes along a longitudinal axis.

Embodiment 9: The connection detecting system of Embodiment 1, wherein the retainer cap is configured to snap fit to the port body via tabs that slip into respective cutouts in the port body.

Embodiment 10: The connection detecting system of Embodiment 1, wherein one or more lenses are placed in between end faces of the pair of light pipes, the emitter, and the detector.

Embodiment 11: The connection detecting system of Embodiment 1, wherein: the emitter and the detector are mounted to a PCB with an aperture through which the port is disposed; and the light is propagated through a pair of light apertures matching a size of the emitter and the detector mounted to the PCB.

Embodiment 12: The connection detecting system of Embodiment 1, wherein: the emitter is a light-emitting diode (LED) configured to generate the light, wherein the emitter is a narrow beam type LED (e.g., ±7° angle of half intensity); and the detector is a photodiode or a phototransistor configured to detect the light, wherein the detector is a wider angle photodiode or a wider angle phototransistor (e.g., ±15° angle of half intensity).

Embodiment 13: The connection detecting system of Embodiment 1, wherein: the emitter generates the light in an IR wavelength; and the detector works in the IR wavelength and filters out other wavelengths (e.g., visible light).

Embodiment 14: The connection detecting system of Embodiment 1, further comprising: an RFID reader configured to detect and read an RFID tag of the surgical device.

Embodiment 15: The connection detecting system of claim 14, wherein: the controller is connected to the RFID reader; and the controller is further configured to determine whether the surgical device is connected to the port based on whether the RFID tag is detected and/or read by the RFID reader.

Embodiment 16: The connection detecting system of Embodiment 1, wherein the emitter and the detector incorporate an RFID reader.

Embodiment 17: The connection detecting system of Embodiment 16, wherein the RFID reader is configured to stay on continuously and provide information of a presence of the connector of the surgical device and/or an identification (ID) of the surgical device.

Embodiment 18: The connection detecting system of Embodiment 16, wherein the detector is configured to determine whether proper connection is established or not based on whether the light is detected.

Embodiment 19: The connection detecting system of Embodiment 16, wherein the RFID reader is configured to determine whether a correct surgical device is connected based on the RFID reading.

Embodiment 20: The connection detecting system of Embodiment 16, wherein the RFID reader is configured to stay off, and only turns on when the detector determines proper connection is established based on whether the light is detected, and wherein the RFID reader is configured to confirm a correct surgical device is connected.

Embodiment 21: The connection detecting system of Embodiment 20, wherein the RFID reader subsequently turns off until a next cycle of the detector changing a state from detecting light to not detecting light.

Embodiment 22: A connection detecting system for a surgical console, the surgical console comprising: a port for receiving a connector of a surgical device, the port comprising: a port body with apertures; a light transmission system comprising: a detection zone through which the connector of the surgical device is configured to be disposed when the surgical device is coupled to the surgical console; and a pair of light pipes disposed in the apertures of the port body at opposite ends of the detection zone, wherein a longitudinal axis of each of the light pipes is orthogonal to a longitudinal axis of the port; and a detector configured to detect the light propagated through the light transmission system; and a controller, connected to the emitter and the detector, configured to determine whether the surgical device is connected to the port based on whether the light is detected by the detector.

Embodiment 23: The connection detecting system of Embodiment 22, wherein: the emitter is a light-emitting diode (LED) configured to generate the light, wherein the emitter is a narrow beam type LED (e.g., ±7° angle of half intensity); and the detector is a photodiode or a phototransistor configured to detect the light, wherein the detector is a wider angle photodiode or a wider angle phototransistor (e.g., ±15° angle of half intensity).

Embodiment 24: The connection detecting system of Embodiment 22, wherein: the emitter generates the light in an IR wavelength; and the detector works in the IR wavelength and filters out other wavelengths (e.g., visible light).

Embodiment 25: The connection detecting system of Embodiment 22, wherein each of the pair of light pipes comprises: two sections of larger diameters and smaller diameters, which allow a sealing ring seated on the smaller diameter.

Embodiment 26: The connection detecting system of Embodiment 25, wherein the pair of light pipes are press fit in body apertures of the port body, which hold the pair of light pipes and each of the sealing rings in position and seal against pneumatic pressure of the port.

Embodiment 27: The connection detecting system of Embodiment 22, further comprising: an RFID reader configured to detect and read an RFID tag of the surgical device.

Embodiment 28: The connection detecting system of claim 27, wherein: the controller is connected to the RFID reader; and the controller is further configured to determine whether the surgical device is connected to the port based on whether the RFID tag is detected and/or read.

Embodiment 29: The connection detecting system of Embodiment 22, wherein the emitter and the detector incorporate an RFID reader.

Embodiment 30: The connection detecting system of Embodiment 29, wherein the RFID reader is configured to stay on continuously and provide information of a presence of the connector of the surgical device and/or its identification.

Embodiment 31: The connection detecting system of Embodiment 29, wherein the detector is configured to determine whether proper connection is established or not based on whether the light is detected.

Embodiment 32: The connection detecting system of Embodiment 29, wherein the RFID reader is configured to determine whether a correct surgical device is connected based on the RFID reading.

Embodiment 33: The connection detecting system of Embodiment 29, wherein the RFID reader is configured to stay off, and only turns on when the detector determines proper connection is established based on whether the light is detected, and wherein the RFID reader is configured to confirm a correct surgical device is connected.

Embodiment 34: The connection detecting system of Embodiment 33, wherein the RFID reader subsequently turns off until a next cycle of the detector changing a state from detecting light to not detecting light.

While the foregoing is directed to embodiments of the present disclosure, other and further embodiments of the disclosure may be devised without departing from the basic scope thereof, and the scope thereof is determined by the claims that follow.

What is claimed is:

1. A connection detecting system for a surgical console, the connection detecting system comprising:
a port for receiving a connector of a surgical device, the port comprising:
a light transmission system comprising:
a detection zone through which the connector of the surgical device is configured to be disposed when the surgical device is coupled to the surgical console; and
a pair of light pipes disposed at opposite ends of the detection zone,
wherein a longitudinal axis of each of the light pipes is parallel relative to a longitudinal axis of the port; and
an emitter configured to:
generate a light for propagation through the light transmission system; and
a detector configured to:
detect the light propagated through the light transmission system; and
a controller, connected to the emitter and the detector, configured to determine whether the surgical device is connected to the port based on whether the light is detected by the detector.

2. The connection detecting system of claim 1, wherein the connector of the surgical device is configured to block the propagation of the light through the detection zone of the light transmission system when properly connected.

3. The connection detecting system of claim 1, wherein the detector is configured to detect the light when the connector of the surgical device is not connected or is improperly connected.

4. The connection detecting system of claim 1, wherein the detection zone is disposed across a coupling zone of the port.

5. The connection detecting system of claim 1, wherein the detection zone is disposed across a peripheral edge of a coupling zone of the port.

6. The connection detecting system of claim 1, wherein the detection zone is disposed across a center of a coupling zone of the port.

7. The connection detecting system of claim 1, wherein each of the light pipes comprises:
an elongated body with a first end face; and
an angled end with:
a second end face substantially parallel to a longitudinal axis of the elongated body; and
an angled surface that is substantially 45 degrees relative to the longitudinal axis of the elongated body, the angled surface configured to redirect the light propagated thereon.

8. The connection detecting system of claim 7, wherein:
the first end face of the elongated body is disposed against a printed circuit board (PCB); and
the second end face of the angled end is disposed above a base surface of a coupling zone of the port,
wherein the light is configured to be propagated across the base surface of the coupling zone.

9. The connection detecting system of claim 1, wherein:
the emitter and the detector are mounted to a PCB; and
the light is propagated and detected through the PCB.

10. The connection detecting system of claim 1, wherein:
the emitter is a light-emitting diode (LED) emitter configured to generate the light; and
the detector is a photodiode or a phototransistor configured to detect the light.

11. The connection detecting system of claim 1, wherein the light is an infrared (IR) light.

12. The connection detecting system of claim 1, further comprising:
a Radio-Frequency Identifier (RFID) reader configured to detect an RFID tag of the surgical device.

13. The connection detecting system of claim 12, wherein:
the controller is connected to the RFID reader; and
the controller is further configured to determine whether the surgical device is connected to the port based on whether the RFID tag is detected.

14. A connection detecting system for a surgical console, the connection detecting system comprising:
a port for receiving a connector of a surgical device, the port comprising:
a light transmission system comprising:
a detection zone through which the connector of the surgical device is configured to be disposed when the surgical device is coupled to the surgical console; and
a pair of light pipes disposed at opposite ends of the detection zone,
wherein a longitudinal axis of each of the light pipes is orthogonal to a longitudinal axis of the port; and
an emitter configured to:
generate a light for propagation through the light transmission system; and
a detector configured to:
detect the light propagated through the light transmission system; and
a controller, connected to the emitter and the detector, configured to determine whether the surgical device is connected to the port based on whether the light is detected by the detector.

15. The connection detecting system of claim 14, wherein:
the emitter and the detector are mounted to a printed circuit board (PCB) with an aperture through which the port is disposed; and
the emitter is configured to propagate the light across the aperture.
